(19) United States
(12) Patent Application Publication
(10) Pub. No.: US 2025/0001129 A1
(43) Pub. Date: Jan. 2, 2025

(54) ENDOVASCULAR DEVICES WITH EXPANDABLE FILTER AND SHEATH

(71) Applicant: Daniel Ezra Walzman, Teaneck, NJ (US)

(72) Inventor: Daniel Ezra Walzman, Teaneck, NJ (US)

(21) Appl. No.: 18/786,877

(22) Filed: Jul. 29, 2024

Related U.S. Application Data
(63) Continuation of application No. 18/724,219, filed on Jan. 1, 0001.
(60) Provisional application No. 63/472,855, filed on Jun. 14, 2023.

(51) Int. Cl. A61M 25/00; A61F 2/24; A61M 25/01

(52) U.S. Cl. CPC A61M 25/0023; A61F 2/2427; A61M 25/0045; A61M 25/005; A61M 25/0074; A61M 25/0105; A61M 2025/0024; A61M 2025/0025; A61M 2025/0047; A61M 2025/0062; A61M 2205/0216; A61M 2205/0222; A61M 2205/0238; A61M 2205/025; A61M 2205/7545

(57) ABSTRACT

A catheter with a filter that is configured for use in treating vascular abnormalities. The filter element is configured to capture emboli within a patient's blood vessel and is expandable from a collapsed configuration and can have a stretchable proximal portion to accommodate insertion of an endovascular treatment device. The catheter is configured for radial expansion to thereby facilitate insertion of an endovascular treatment device into the catheter lumen, whereby the catheter is reconfigured from a normal configuration, in which the main lumen defines a first dimension, into an expanded configuration, in which the main lumen defines a second dimension larger than the first dimension.

### BACKGROUND OF THE INVENTION

This application claims priority to provisional application Ser. No. 63/472,855, filed Jun. 14, 2023, the entire contents of which are incorporated herein by reference.

### TECHNICAL FIELD

The present disclosure provides various systems for treating vascular abnormalities (e.g., blockages, constrictions, and the like) that include one or more medical devices (e.g., catheters, tubular members, and other such elongated members), which may be employed individually or in combination with each other, as well as methods of using the same during the course of an endovascular procedure.

### BACKGROUND

The direct and indirect adverse health effects associated with the presence of solid matter (e.g., a thrombus) within an individual's vascular system is well documented, which has led to the development of various endovascular systems and methods of treatment. An opportunity remains, however, for improvements in the treatment of such vascular abnormalities.

### SUMMARY

In one aspect of the present invention, an endovascular system is disclosed that is configured for insertion into a patient's blood vessel. The endovascular system includes a first tubular member having a main lumen and being configured for radial expansion to thereby enlarge the main lumen upon insertion of a medical device and reconfigure the first tubular member from a first configuration, in which the main lumen defines a first dimension, into a second configuration, in which the main lumen defines a second dimension larger than the first inner cross-sectional dimension.

In some embodiments, the first tubular member may be substantially resilient in construction. The first tubular member in some embodiments may include rubber, latex or spandex.

In certain embodiments, the first tubular member may be configured to return to the first configuration following removal of the medical device.

In certain embodiments, the first tubular member may be reconfigured from the second configuration into a third configuration following removal of the medical device. In some embodiments, the first tubular member may define a third dimension in the third configuration larger than the first dimension but smaller than the second dimension.

In certain embodiments, the endovascular system may further include a filter element that is supported by the first tubular member. The filter element may include a semi-permeable membrane. In some embodiments, the filter element may be reconfigurable between a collapsed configuration and an expanded configuration to capture emboli within the patient's blood vessel.

In some embodiments, at least a portion of the filter element may include a stretchable material to enable expansion of the filter element during passage of the medical device through the filter element.

In certain embodiments, the filter element may include a proximal portion attached to the first tubular member and a distal portion. In some embodiments, at least the proximal portion of the filter element may be expandable. In some embodiments, the proximal portion of the filter element may include a first material and the distal portion of the filter element may include a second material. The first material and the second material may have substantially equivalent resiliencies. In some embodiments, the second material may be less resilient than the first material.

In some embodiments, the filter element and the first tubular member may include at least one common resilient material.

In some embodiments, the endovascular system may further include a positioning member including a non-annular cross-sectional configuration.

In some embodiments, the medical device may include a replacement valve.

In some embodiments, the first tubular member may include at least one steerable segment. In some embodiments, the first tubular member may be configured for rotational deflection to thereby vary an angular position of the first tubular member.

In another aspect of the present invention, an endovascular system is disclosed that is configured for insertion into a patient's blood vessel. The endovascular system includes a first tubular member and a filter element that defines a passage, which extends therethrough. The filter element includes a proximal portion that is attached to the first tubular member and a distal portion. The filter element includes a semi-permeable membrane and is reconfigurable from a collapsed configuration into an expanded configuration to capture emboli within the patient's blood vessel, wherein at least the proximal portion of the filter element includes a first stretchable material to accommodate insertion of a medical device into the passage.

In some embodiments, the medical device may include a replacement valve.

In some embodiments, the first tubular member may include a resilient construction. The first tubular member may include rubber or spandex. The first tubular member may include a second stretchable material. In some embodiments, the first stretchable material may be less resilient than the second stretchable material.

In another aspect of the present disclosure, an endovascular system is disclosed that includes a first member having a filter element with a semi-permeable membrane attached thereto and a second member that is insertable alongside the first member. The filter element is expandable into contact with the second member and is reconfigurable between a collapsed configuration and an expanded configuration to capture emboli.

The second member in some embodiments, may include an annular configuration or a non-annular configuration. The second member can at least one flattened section. In some embodiments, the second member may include an elliptical cross-sectional configuration.

In certain embodiments, the second member may include a body defining a lumen. In some embodiments, the body and the lumen may include dissimilar configurations.

In another aspect of the present invention, a catheter is disclosed that is configured for use in treating vascular abnormalities. The catheter defines a main lumen and includes a filter element that is configured to capture emboli within a patient's blood vessel. The catheter is configured for radial expansion to thereby facilitate insertion of an endovascular treatment device into the main lumen, whereby the catheter is reconfigured from a normal configuration, in which the main lumen defines a first dimension, into an expanded configuration, in which the main lumen defines a second dimension larger than the first dimension.

In some embodiments, the catheter may include at least one guide rail that is located within the main lumen and which is configured for engagement with the endovascular treatment device to facilitate insertion of the endovascular treatment device into the catheter and advancement of the endovascular treatment device through the main lumen.

In some embodiments, the catheter may include at least one strengthening rib that is configured to inhibit variation in an overall length of the catheter during radial expansion.

In some embodiments, the catheter may include a non-resilient construction such that the expanded configuration is generally maintained following removal of the endovascular treatment device. In some embodiments, the catheter may include a resilient construction such that the catheter is biased towards the normal configuration.

In some embodiments, the catheter may include at least one stretchable material. The catheter in some embodiments, may include at least one of rubber, latex, and spandex.

In certain embodiments, the catheter may be configured to return to the normal configuration following removal of the endovascular treatment device.

In certain embodiments, the catheter may be configured such that, following removal of the endovascular treatment device, the catheter is reconfigured into a partially-expanded configuration in which the main lumen defines a third dimension larger than the first dimension and smaller than the second dimension.

In some embodiments, the catheter may be configured such that the third dimension lies substantially within the range of approximately 105% to approximately 195% of the first dimension.

In another aspect of the present invention, an endovascular system is disclosed that includes a main catheter defining a main lumen and an inner catheter that extends through the main lumen. The main catheter includes a filter element that is reconfigurable from a collapsed configuration, in which the main catheter is configured for insertion into a patient's blood vessel, into an expanded configuration, in which the filter element is configured to capture emboli within the patient's blood vessel. The inner catheter is configured for axial movement in relation to the main catheter from a retracted position into an advanced position to thereby facilitate reconfiguration of the filter element from the collapsed configuration into the expanded configuration.

In some embodiments, the inner catheter may include at least one area of increased thickness such that the inner catheter defines a non-uniform transverse cross-sectional dimension to thereby increase rigidity, strength, and/or stability of the inner catheter.

In some embodiments, the endovascular system may further include an outer sheath that is configured to receive the main catheter such that the outer sheath extends about the main catheter. In some embodiments, the main catheter and the outer sheath may be configured for relative axial movement.

In some embodiments, the inner catheter may include a retainer that is configured to receive the filter element. The retainer may extend about the filter element when the inner catheter is in the retracted position to thereby constrain the filter element and preserve the collapsed configuration thereof. The retainer may be located distally beyond the filter element when the inner catheter is in the advanced position to allow for reconfiguration of the filter element from the collapsed configuration into the expanded configuration.

In some embodiments, the retainer may be configured to entirely conceal the filter element when the inner catheter is in the retracted position; in other embodiments, the retainer may be configured to partially conceal the filter element when the inner catheter is in the retracted position.

In some embodiments, the retainer may be connected to the filter element. The retainer may be connected to the filter element via at least one frangible member that is configured to inhibit relative movement between the inner catheter and the main catheter until a sufficient axial force is applied thereto. The at least one frangible member may be configured to rupture during movement of the inner catheter from the retracted position into the advanced position.

In some embodiments, the retainer may be reconfigurable from an enlarged configuration into a compressed configuration during movement of the inner catheter from the retracted position into the advanced position.

In some embodiments, the retainer may include a resilient construction such that the retainer is biased towards the compressed configuration, whereby the retainer is automatically reconfigured from the enlarged configuration into the compressed configuration upon movement of the inner catheter from the retracted position into the advanced position.

In some embodiments, the retainer may include at least one stretchable material.

In some embodiments, the inner catheter may include a cinching mechanism to facilitate reconfiguration of the retainer from the enlarged configuration into the compressed configuration. The inner catheter may further include at least one extension that is secured to the retainer such that the at least one extension extends proximally from the retainer and covers the filter element when the inner catheter is in the retracted position. The inner catheter may include a plurality of extensions arranged in an overlapping configuration.

In some embodiments, the at least one extension may be biased radially inward such that the at least one extension is automatically deflected radially inward upon movement of the inner catheter from the retracted position into the advanced position. The at least one extension may include a resilient construction.

In certain embodiments, the inner catheter may include a cinching mechanism to deflect the at least one extension radially inward.

### BRIEF DESCRIPTION OF THE DRAWINGS

According to common practice, the various features of the drawings may not be to scale and may be arbitrarily expanded or reduced for clarity.

FIG. 1 illustrates an endovascular system including a main catheter with a filter element according to one embodiment of the present invention, which is shown inserted into a patient's blood vessel during an endovascular procedure.

FIG. 2 is a transverse, cross-sectional view of the main catheter according to one embodiment of the present invention.

FIG. 3 is a transverse, cross-sectional view of the main catheter taken along line 3-3 in FIG. 1, which is shown in a first configuration with various embodiments of endovascular treatment devices that are configured for insertion into the main catheter.

FIG. 4A is a transverse, cross-sectional view of the main catheter shown in a second configuration upon insertion of the endovascular treatment device.

FIG. 4B is a side view of the main catheter shown in a second configuration upon insertion of the endovascular treatment device.

FIG. 5 is a transverse, cross-sectional view of the main catheter shown in a third configuration following removal of the endovascular treatment device.

FIG. 6 is a transverse, cross-sectional view of the main catheter according to one embodiment of the present invention in which the main catheter includes one or more strengthening ribs.

FIG. 7 is a transverse, cross-sectional view of the main catheter according to one embodiment of the present invention in which the main catheter includes one or more guide rails.

FIG. 8 is a partial, top, plan view of the main catheter with the filter element shown in a collapsed configuration.

FIG. 9 is a partial, top, plan view of the main catheter with the filter element of FIG. 8 shown in an expanded configuration.

FIG. 10 illustrates one embodiment of the endovascular system including a positioning member that is configured to direct the filter element into engagement with the blood vessel during the endovascular procedure.

FIG. 11 illustrates an alternate method of using the endovascular system seen in FIG. 10.

FIG. 12 is a transverse, cross-sectional view of the positioning member taken along line 12-12 in FIG. 10.

FIG. 13 is a transverse, cross-sectional view of the positioning member according to one embodiment of the present invention.

FIG. 14 is a transverse, cross-sectional view of the positioning member according to one embodiment of the present invention.

FIG. 15 is a transverse, cross-sectional view of the positioning member according to one embodiment of the present invention.

FIG. 16 illustrates an alternate embodiment of the endovascular system including an outer sheath that extends about the main catheter.

FIG. 17 is a transverse, cross-sectional view of the outer sheath and the main catheter taken along line 17-17 in FIG. 16.

FIG. 18 is a partial, proximal, perspective view of the outer sheath shown with a series of stylets.

FIG. 19 is a partial, plan view of the main catheter according to one embodiment of the present disclosure including one or more steerable segments and shown in a first configuration.

FIG. 20 is a transverse, cross-sectional view of the main catheter taken along line 20-20 in FIG. 19.

FIG. 21 is a partial, plan view of the main catheter seen in FIG. 19 shown in a second configuration.

FIG. 22 is a partial, perspective view of the main catheter according to one embodiment of the present disclosure in which the main catheter is configured for rotational deflection.

FIG. 23 illustrates one embodiment of the endovascular system including an inner catheter with a retainer that extends through the main catheter, which is shown in a retracted position.

FIG. 24 illustrates the endovascular system seen in FIG. 23 with the inner catheter shown in an advanced position.

FIG. 25 illustrates the endovascular system seen in FIG. 23 with the inner catheter according to one embodiment of the present invention.

FIG. 26 illustrates the endovascular system seen in FIG. 23 with the inner catheter according to one embodiment of the present invention, which includes one or more extensions that extend proximally from the retainer.

### DETAILED DESCRIPTION OF THE INVENTION

The present disclosure describes various devices, systems, and methods for treating vascular abnormalities (e.g., blockages, constrictions, and the like) in a patient's leg(s), arm(s), torso, neck, head, etc. For example, the present disclosure describes a main catheter (also referred to herein as a sheath or a tubular member) that is configured for radial expansion to thereby facilitate insertion of an endovascular treatment device. In order to capture emboli within a patient's blood vessel, the catheter (sheath/tubular member) includes a filter element having a semi-permeable membrane that is reconfigurable between a collapsed configuration and an expanded configuration.

The expandable sheath can also be used as one piece (“1st piece”) of a multiport filter system for capturing and removing clot, debris, etc. Uses include, but are not limited to, capturing clots during a thrombectomy procedure (for example, placed from the neck/internal jugular vein or subclavian vein, with the filter in the inferior vena cava, during an iliac/lower extremity and/or lower IVC thrombectomy, and/or placed from the femoral vein, with the filter in a vein rostral to heart, in upper extremity, and/or head/neck thrombectomy for dvt). It can also be used from artery access (most of transfemoral) to place the filter in ascending aorta during aortic valve replacement, and/or other left heart procedures.

In certain embodiments, the main catheter may be provided as a component of an endovascular system that also includes a positioning member, which is insertable alongside the main catheter. During use of the endovascular system, upon reconfiguration of the filter element from the collapsed configuration into the expanded configuration, the filter element contacts the positioning member and is forced into apposition with the blood vessel.

Additionally, or alternatively, the endovascular system may include an outer sheath that is configured to receive the main catheter (e.g., to facilitate delivery of the main catheter and the filter element) and/or an inner catheter that extends through, and is configured for axial movement in relation to, the main catheter. More specifically, in such embodiments, the inner catheter is repositionable between a retracted position and an advanced position to thereby facilitate reconfiguration of the filter element between the collapsed configuration and the expanded configuration.

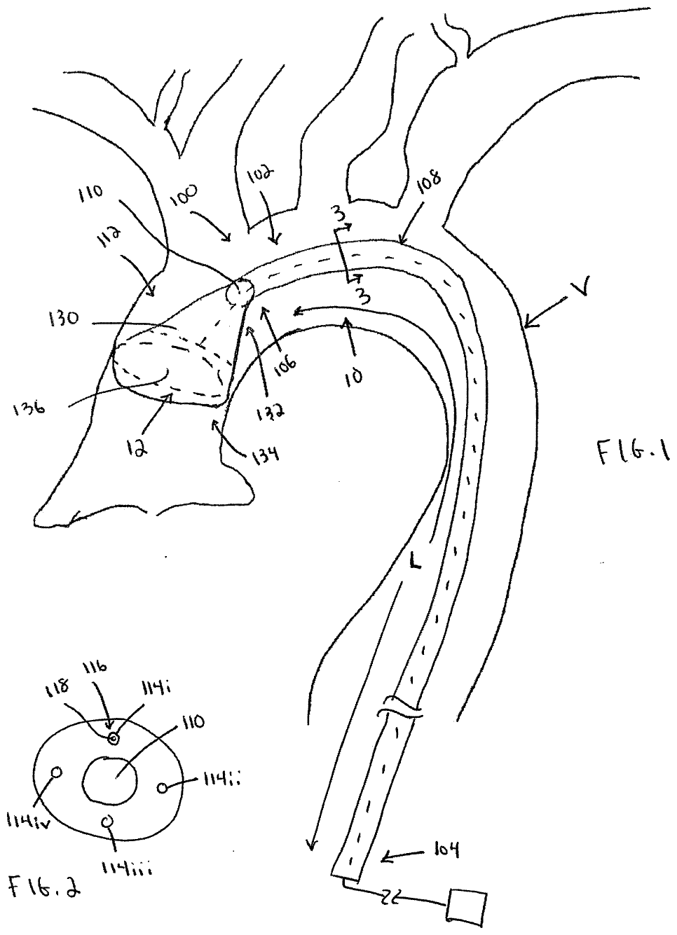

With reference to FIG. 1, an endovascular system 10 is illustrated that is configured for use in treating vascular abnormalities. The endovascular system 10 is configured for insertion into a patient's blood vessel V and includes a (first) endovascular treatment (medical) device 100, which is configured as a (first) main catheter (tubular member) 102 defining a length L and having: respective proximal and distal portions (ends) 104, 106; an outer wall 108 that that defines a main (central, working) lumen 110, which extends continuously between the respective proximal and distal portions 104, 106 of the main catheter 102; and a filter element 112, which is supported by (e.g., at or adjacent to) the distal portion 106 of the main catheter 102.

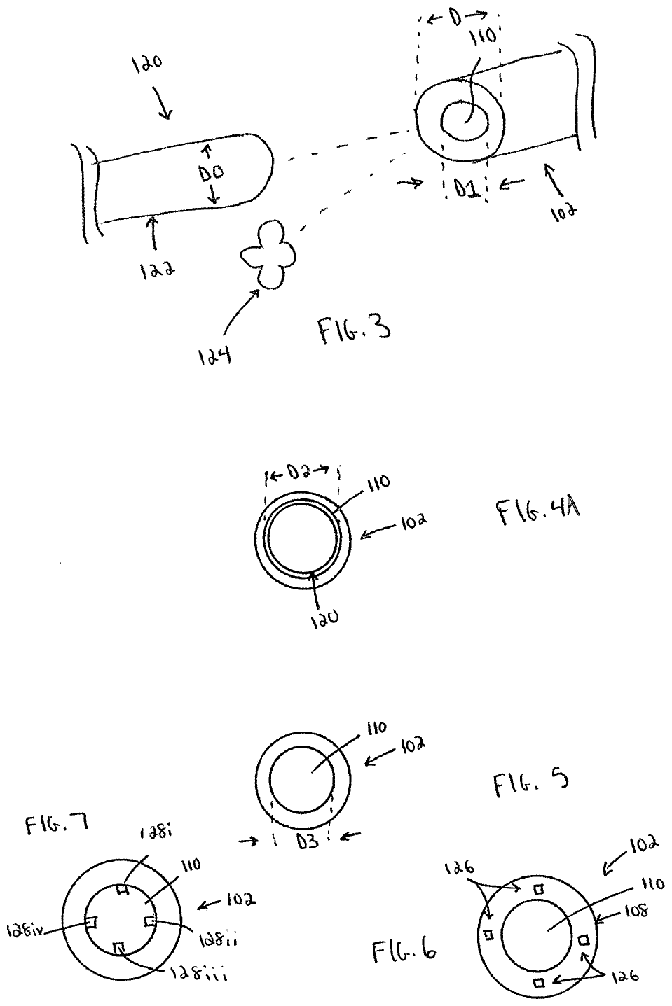

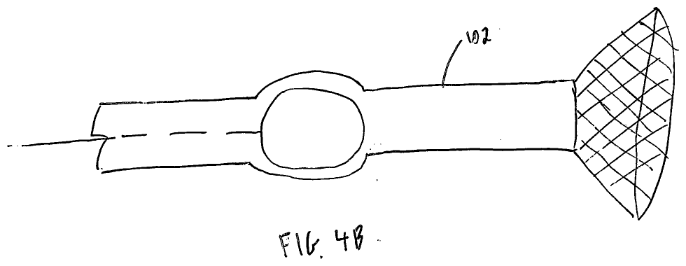

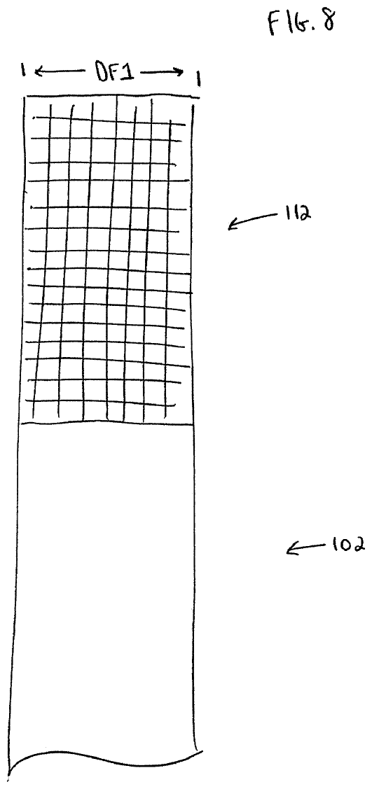

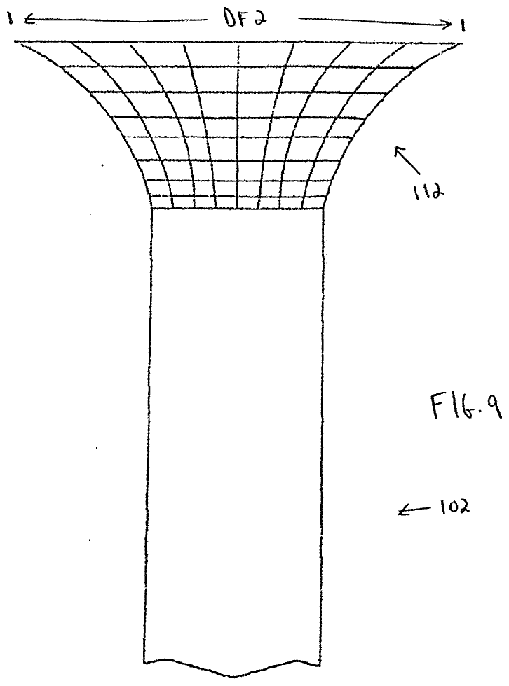

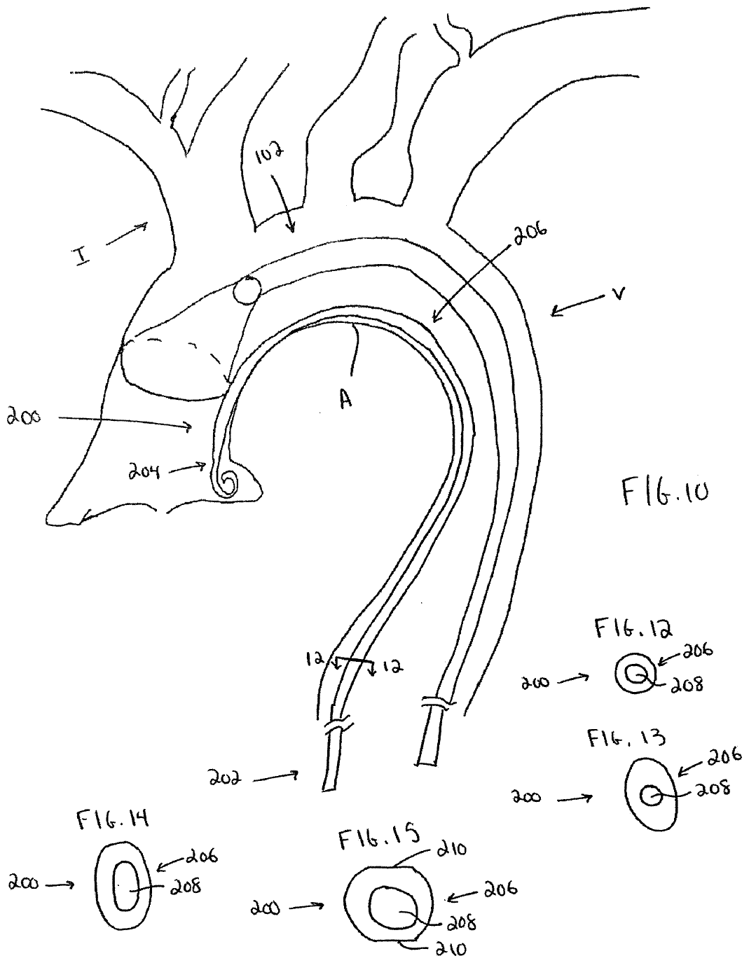

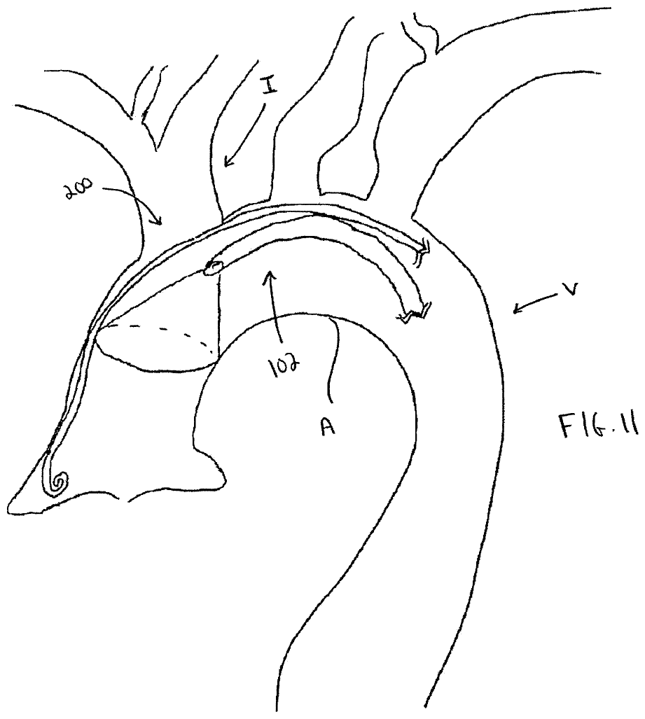

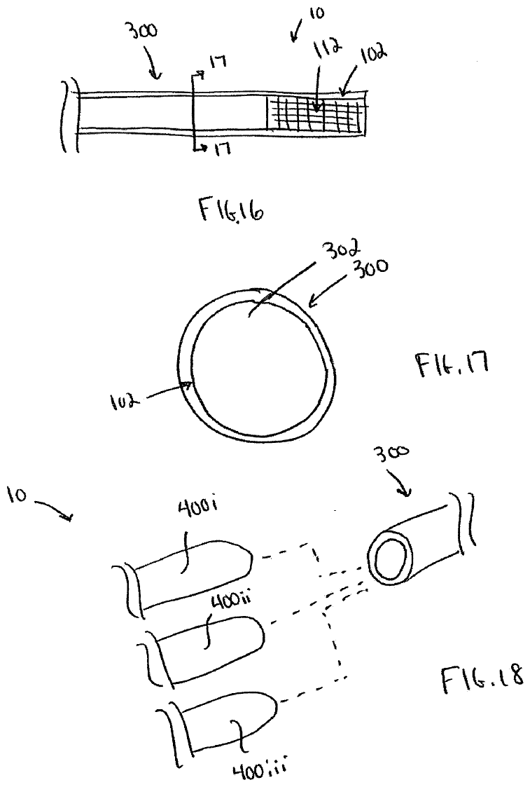

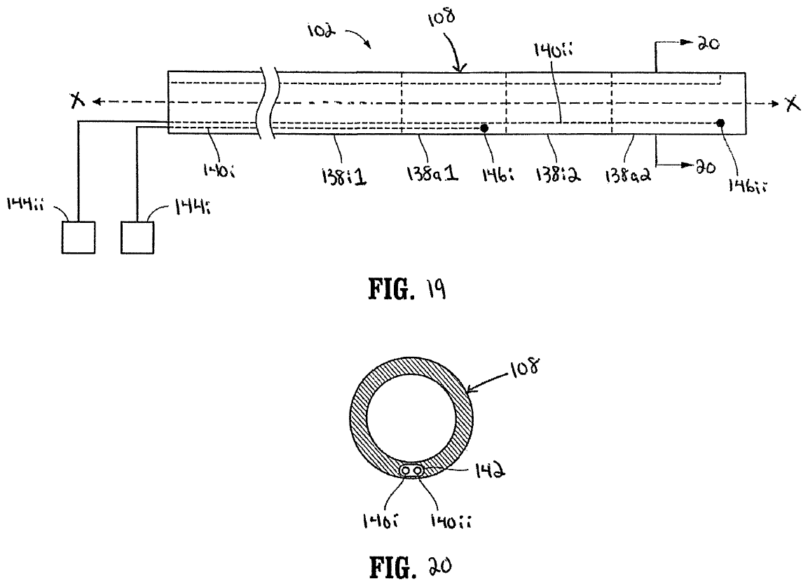

While the outer wall 108 of the main catheter 102 is illustrated as being solid in the embodiment illustrated in FIG. 1, embodiments are also envisioned in which the outer wall 108 may include (at least one) one or more internal channels 114 that are configured to receive a supplemental (additional) medical device 116 (e.g., a guidewire 118, an additional catheter, etc.), as seen in FIG. 2. Although shown as including four internal channels 114i-114iv in the embodiment illustrated in FIG. 2, it should be appreciated that the particular number of channels 114 may be increased or decreased without departing from the scope of the present invention. As such, embodiments including both greater and fewer numbers of internal channels 114 are envisioned herein and would not be beyond the scope of the present invention.

The main lumen 110 defines and a (first, initial) inner transverse cross-sectional dimension (e.g., a diameter) D1 (FIG. 3) and is configured to receive a (second) endovascular treatment (medical) device 120 (e.g., a (second) catheter 122, a replacement valve 124, thrombectomy device, etc.) having an outer transverse cross-sectional dimension (e.g., a diameter) DO that is larger than the inner transverse cross-sectional dimension D1, as described in further detail below. In the illustrated embodiment, the main catheter 102 is configured such that the inner transverse cross-sectional dimension D1 lies substantially within the range of (approximately) 0.05 French to (approximately) 32 French. Embodiments in which the inner transverse cross-sectional dimension D1 may lie outside the disclosed range, however, are also contemplated herein and would not be beyond the scope of the present invention. Note the main lumen can be circular in transverse dimension or other non-circular shapes, e.g., oval.

In certain embodiments, the main catheter 102 may be configured for radial expansion (e.g., upon insertion of the (second) endovascular treatment device 120) to thereby enlarge the main lumen 110 and reconfigure the catheter from a first (normal, contracted) configuration (FIG. 3) into a second (expanded, enlarged) configuration (FIGS. 4A and 4B). More specifically, in the first configuration, the main lumen 110 defines the inner transverse cross-sectional dimension DI, and in the second configuration, the main lumen 110 defines a (second, subsequent) inner transverse cross-sectional dimension (e.g., a diameter) D2, which is larger than the inner transverse cross-sectional dimension D1 and (approximately) equal to the outer transverse cross-sectional dimension DO of the (second) endovascular treatment device 120. Note the catheter 102 can be circular in transverse dimension or other non-circular shapes, e.g., oval. Note FIG. 4B shows insertion of a medical instrument with an enlarged distal region which expands the catheter as it is advanced; in other embodiments, other regions of the catheter can be enlarged like the distal region, e.g., enlarged along a length, so it would expand/stretch other regions of the main catheter as it passes through.

In order to facilitate such reconfiguration of the main catheter 102, the main catheter 102 may include any suitable material or combination of materials. In one embodiment, the main catheter 102 may be non-resilient in construction, whereby the second configuration is (generally) maintained following removal of the (second) endovascular treatment device 120. Alternatively, however, the main catheter 102 may be resilient in construction such that the main catheter 102 is biased towards the first configuration, whereby the main catheter 102 automatically moves towards the first configuration (FIG. 3) following removal of the (second) endovascular treatment device 120. For example, the main catheter 102 may include (e.g., may be formed partially or entirely from) (at least one) one or more resilient (expandable, stretchable) materials such as, for example, rubber, latex, resin and/or spandex.

The stretchy resin or other material may have a braid (or strand) of wires or a braid (or strand) of PET or other non-metal fibers, or a braid where some strands are metal and some are non-metal, e.g., PET, in various ratios. The braid fibers may protrude slightly from the resin along the inner diameter, which can act as “rails” for things to slide on (e.g., devices, tissue, etc.), in order to minimize friction from the stretchy material surface. Fibers also can have the braid change configuration, without the fibers being elongated/stretched, so this creates less friction that something where a stretching force is distorting the structure.

In some embodiments, an inner hydrophilic coating is provided on the fibers/strands or other materials to reduce friction.

In such embodiments, following removal of the (second) endovascular treatment device 120, the main catheter 102 may return to the first configuration (FIG. 3). Alternatively, following removal of the (second) endovascular treatment device 120, the main catheter 102 may be reconfigured into a third (partially-expanded) configuration in which the main lumen 110 defines a (third) inner transverse cross-sectional dimension (e.g., a diameter) D3 (FIG. 5) that is larger than the inner transverse cross-sectional dimension D1 (FIG. 3) and smaller than the inner transverse cross-sectional dimension D2 (FIG. 4). Depending upon the specific material(s) utilized in construction of the main catheter 102, it is envisioned that D3 may lie (substantially) within the range of (approximately) 105% to (approximately) 195% of D1.

In some embodiments, the main catheter 102 may be configured such that an outer transverse cross-sectional dimension (e.g., a diameter) D thereof remains (generally) constant during expansion (and contraction) of the main catheter 102 (e.g., during reconfiguration of the main catheter 102 from the first configuration (FIG. 3) into the second configuration (FIG. 4) and during reconfiguration of the main catheter 102 from the second configuration into the first configuration). Thus, in these embodiments, the inner lumen expands (reducing the wall thickness of the catheter), but the outer diameter remains constant. In other embodiments, however, it is envisioned that the main catheter 102 may be configured such that the outer transverse cross-sectional dimension D varies during axial expansion (and contraction) of the main catheter 102. More specifically, the main catheter 102 may be configured such that the outer transverse cross-sectional dimension D is enlarged during reconfiguration of the main catheter 102 from the first configuration into the second configuration and such that the outer transverse cross-sectional dimension D is reduced during reconfiguration of the main catheter 102 from the second configuration into the first configuration.

The main catheter 102 can recoil back to various configurations/sizes after passage of the medical device therethrough, e.g., after removal of the medical device from the main lumen. In some embodiments, it can recoil back to at least 50% of its original configuration. In other embodiments, it can recoil to at least 80% and in other embodiments to at least 90% of its original configuration. In other embodiments, it can recoil to greater than 90% or greater than 95%. Recoil back to other percentages between the range of about 50% to about 100% (complete recoil) are also contemplated, e.g. In some embodiments, the main catheter can recoil to less than 50% of its original size. Thus, the catheter (sheath/tubular member) is capable of expanding from a normal inner diameter when a larger device is passed through it, to allow passage of the device therethrough, and has recoil/memory to spring back to a percentage of the stretched/increased inner/outer diameter.

With reference to FIG. 6, in some embodiments, the main catheter 102 may include (at least one) one or more strengthening ribs 126 in order to inhibit (if not entirely prevent) axial elongation and/or axial contraction (e.g., variation in the length L (FIG. 1) of the main catheter 102) during radial expansion (and contraction) (e.g., during insertion and removal of the (second) endovascular treatment device 120). In the illustrated embodiment, the strengthening ribs 126 are embedded (located) within the outer wall 108 of the main catheter 102 and extend axially (longitudinally) along the length L thereof. It should be appreciated, however, that the strengthening ribs 126 may be included in any suitable location (e.g., externally of the main catheter 102 (on the outer wall 108), within the main lumen 110, etc.) and may be configured in any suitable manner. For example, embodiments in which the strengthening ribs 126 extend at acute or right angles to the length L of the main catheter 102 are also envisioned herein, as are embodiments in which the strengthening ribs 126 may extend helically along the length L of the main catheter 102.

The strengthening ribs 126 may include any suitable material or combination of materials, whether similar (e.g., identical) or dissimilar (e.g., non-identical) to those used in construction of the remainder of the main catheter 102 (e.g., the outer wall 108). For example, in one particular embodiment, the strengthening ribs 126 may include (e.g., may be formed partially or entirely from) synthetic fibers (e.g., Kevlar®).

Additionally, or alternatively, the main catheter 102 may include (at least one) one or more guide rails 128 that are located within (extend into) the main lumen 110. The guide rail(s) 128 extend between the respective proximal and distal portions 104, 106 (FIG. 1) of the main catheter 102 and are configured for engagement (contact) with the (second) endovascular treatment device 120 (and/or other devices) to facilitate insertion of the (second) endovascular treatment device 120 (and/or other devices) into the main catheter 102 and advancement of the (second) endovascular treatment device 120 (and/or other devices) through the main lumen 110. Although shown as including four guide rails 128i-128iv in the illustrated embodiment, it should be appreciated that the particular number of guide rails 128 may be increased or decreased. As such, embodiments including both greater and fewer numbers of guide rails 128 are envisioned herein and would not be beyond the scope of the present invention.

In some embodiments, the catheter (sheath/tubular member) can have one or more folded seams (like a Chinese fan) which unfolds when needed to expand to a larger diameter.

Note the ribs and/or guiderails can be provided on the various catheter embodiments disclosed herein.

The main catheter can be in the form of a vascular sheath or port for surgery, (e.g., robotics, laparoscopic, bronchoscopic, GU, GI, etc.). Veterinary uses are also contemplated.

The filter element 112 includes a semi-permeable membrane 130 (FIG. 1) (e.g., a net, a mesh, or other such suitable structure) and is configured to capture emboli or other such debris located within the patient's blood vessel V. As seen in FIG. 1, the filter element 112 includes a proximal portion 132, which is supported by (e.g., connected to) the distal portion 106 of the main catheter 102, and a distal portion 134, which flares radially outward (e.g., in relation to the proximal portion 132) so it has a larger diameter at a distal portion. Additionally, the filter element 112 defines a passage 136, which extends therethrough and is in communication with the main lumen 110 of the main catheter 102 so as to facilitate insertion of the (second) endovascular treatment device 120 (and/or other devices) into (and/or through) the main catheter 102 and the filter element 112 (e.g., via the passage 136) and into the blood vessel V to access a target treatment site. The filter element 112 can be delivered in a compressed state and can either self-expand upon release of a constraint or be mechanically expandable such as a by a balloon.

The filter element 112 may be partially or entirely expandable such that, upon deployment (delivery) of the filter element 112, the filter element 112 is automatically reconfigured from a collapsed (insertion) configuration (FIG. 8), in which the main catheter 102 is configured for insertion into the patient's blood vessel V (FIG. 1) and the filter element 112 defines a (first, initial) transverse cross-sectional dimension (e.g., a diameter) DF1, into an expanded (deployed) configuration (FIGS. 1, 9), in which the filter element 112 defines a (second, subsequent) transverse cross-sectional dimension (e.g., a diameter) DF2 (FIG. 9), which is larger than the transverse cross-sectional dimension DF1. More specifically, as seen in FIG. 1, in the expanded configuration, the filter element 112 engages (contacts) the blood vessel V and is configured to capture emboli within the patient's blood vessel.

In some embodiments, the filter element 112 may be partially or entirely resilient in construction, whereby the filter element 112 is reconfigured from the collapsed configuration (FIG. 8) into the expanded configuration (FIG. 9) upon the insertion of a medical device (e.g., the (second) endovascular treatment device 120 (FIG. 3)) and the filter element 112 is reconfigured from the expanded configuration into the collapsed configuration upon removal of the medical device (e.g., the (second) endovascular treatment device 120). For example, embodiments are envisioned in which at least a portion of the filter element 112 (e.g., the proximal portion 132) may include (at least one) one or more expandable (stretchable) materials (e.g., such that at least the proximal portion 132 of the filter element 112 is expandable) in order to accommodate insertion of the (second) endovascular treatment device 120 into the passage 136.

In certain embodiments, the main catheter 102 and the filter element 112 may include (at least one) one or more common (e.g., similar or identical) resilient (expandable, stretchable) materials of construction. Alternatively, the main catheter 102 (e.g., the outer wall 108) may include a first resilient material and that the filter element 112 may include a second resilient material, which may be more resilient than the first resilient material or less resilient than the first material.

Additionally, or alternatively, the proximal portion 132 of the filter element 112 may include a first material and that the distal portion 134 of the filter element 112 may include a second, different material. For example, the first material and the second material may have substantially equivalent resiliencies. Alternatively, the second material may be less resilient than the first material.

In some embodiments, the expandable catheter/sheath can be one piece with the filter.

With reference now to FIGS. 10 and 11, in certain embodiments, the endovascular system 10 may further include a positioning member 200, which may be utilized to direct the filter element 112 into engagement (contact) with the blood vessel V. More specifically, the positioning member 200 is inserted into the blood vessel V such that the positioning member 200 is located alongside (e.g., in (generally) parallel relation to) the main catheter 102 such that the positioning member 200 is located between the main catheter 102 and the blood vessel V. Depending upon the particular configuration, location, size, etc., of the blood vessel V, the positioning member 200 may be positioned in a variety of orientations. For example, the positioning member 200 may be positioned adjacent to the patient's aortic arch A, as seen in FIG. 10, or the positioning member 200 may be positioned opposite to the patient's aortic arch A (e.g., adjacent to the patient's innominate artery I), as seen in FIG. 11.

Upon insertion of the positioning member 200 and reconfiguration of the filter element 112 into the expanded configuration, the filter element 112 expands into contact with the positioning member 200, whereby the filter element 112 is forced into apposition with the blood vessel V, thus increasing conformity of the filter element 112 with the patient's anatomy and reducing blood flow through the blood vessel V about (around, outside of) the filter element 112. The positioning member 200 thus functions to direct (e.g., increase) blood flow through the filter element 112 as it moves the filter element 112. The positioning member 200 can contact one of both of the filter element 112 and main catheter 102.

The positioning member 200 includes respective proximal and distal portions (ends) 202, 204 and a body 206 that defines a (central) lumen 208 (FIG. 12). In the illustrated embodiment, the positioning member 200 is configured such that the body 206 and the lumen 208 include corresponding (generally) annular (e.g., circular, round) outer and inner transverse cross-sectional configurations, respectively, as seen in FIG. 12. Embodiments in which the body 206 and/or the lumen 208 may include non-annular transverse cross-sectional configurations, however, are also envisioned herein (e.g., to further enhance the ability of the positioning member 200 to direct the filter element 112 into engagement (contact) with the blood vessel V). For example, FIG. 13 illustrates an embodiment of the positioning member 200 in which the outer transverse cross-sectional configuration of the body 206 is (generally) elliptical and the inner transverse cross-sectional configuration of the lumen 208 is (generally) annular, e.g., such that the body 206 and the lumen 208 include non-corresponding (dissimilar) outer and inner transverse cross-sectional configurations, respectively (or other dissimilar configurations), and FIG. 14 illustrates an embodiment of the positioning member 200 in which the outer transverse cross-sectional configuration of the body 206 and the inner transverse cross-sectional configuration of the lumen 208 are each (generally) elliptical (e.g., such that the body 206 and the lumen 208 include corresponding (similar) outer and inner transverse cross-sectional configurations, respectively). FIG. 15 illustrates another embodiment of the positioning member 200 in which the body 206 includes (at least one) one or more flattened (e.g., (generally) planar) sections 210.

It is envisioned that the configuration of the positioning member 200 may be (generally) uniform or variable between the respective proximal and distal portions 202, 204. For example, it the body 206 may include one or more non-annular sections that are located between (generally) annular sections.

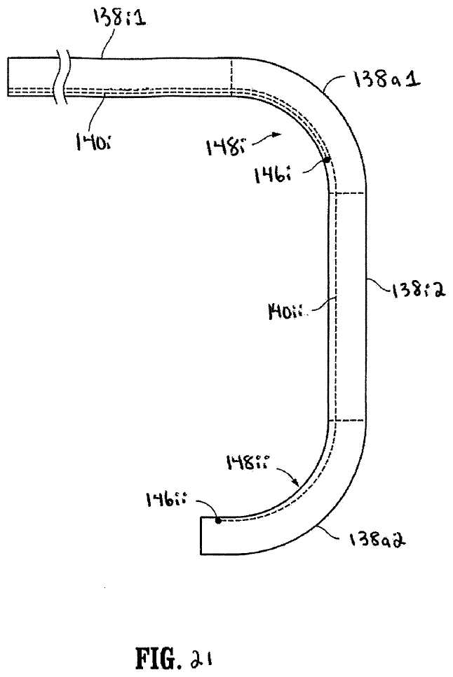

In order to facilitate insertion and removal of the main catheter 102, in some embodiments, the endovascular system 10 may include an outer sheath (tubular member, catheter) 300, as seen in FIG. 16. The outer sheath 300 defines a (primary) lumen 302 that is configured to receive the main catheter 102 such that the outer sheath 300 extends about the main catheter 102, which allows for relative (axial) movement therebetween. In some embodiments, the outer sheath 300 may be configured to collapse the filter element 112 and thereby reconfigure the filter element 112 from the expanded configuration into the collapsed configuration (e.g., to facilitate insertion of the main catheter 102 into the blood vessel V (FIG. 1) and/or removal of the main catheter 102 from the blood vessel V), whereby the outer sheath 300 functions as a restraint (constriction) member (mechanism). Additionally, or alternatively, the endovascular system 10 may include a cinching mechanism 12 (FIG. 1) (e.g., a lasso., a snare, etc.) in order to facilitate reconfiguration of the filter element 112 from the expanded configuration into the collapsed configuration (e.g., prior to removal of the main catheter 102 from the blood vessel V), as described in U.S. patent application Ser. No. 17/210,778 (U.S. Publication No. 2021/0236257) and U.S. Pat. Nos. 11,439,492 and 11,877,752, which are each hereby incorporated by reference in their entirety. In some embodiments a compression mechanism such as an outer sheath can be provided to compress the flared filter for removal from the body, wherein the compression mechanism comprises at least one wire which courses substantially through a segment a wall of the catheter (primary tubular member). In some embodiments, at least one wire courses through a segment of the wall of the catheter and at least partially around a circumference of the filter. A mechanism to move the at least one wire can be provided at a proximal end of the outer tubular member, proximal being on an end opposite the filter which is at the distal end. The mechanism can be activated from outside the patient's body and is actuable, e.g., movable, to collapse the filter by applying a force to move the at least one wire.

The outer sheath 300 may include (at least one) one or more secondary or tertiary lumens (e.g., in addition to the lumen 302). Additionally, or alternatively, the endovascular system 10 may include (at least one) one or more stylets 400 (FIG. 18) that are configured for removable insertion into the outer sheath 300 to facilitate insertion thereof into the blood vessel V (FIG. 1). For example, it is envisioned that the stylets 400 may have varying stiffness (e.g., malleability), and that the stylets 400 may be provided in addition to the main catheter 102 (FIGS. 16, 17) and the outer sheath 300 as a kit. Although shown as including three stylets 400i-400iii in the embodiment illustrated in FIG. 18, it should be appreciated that the particular number of stylets 400 included in the endovascular system 10 may be increased or decreased without departing from the scope of the present disclosure. As such, embodiments including both greater and fewer numbers of stylets 400 are envisioned herein and would not be beyond the scope of the present invention.

It is envisioned that the various devices described herein may (optionally) include (at least one) one or more steerable segments (zones) that are deflectable via (at least one) one or more pull wires that extend (are embedded) within the walls thereof such that the devices are reconfigurable between a variety of configurations. Additionally (or alternatively the various devices described herein may (optionally) include an integrated visualization device or system (e.g., a camera or the like) to facilitate imaging during the course of a surgical procedure. For example, with reference to FIGS. 19-21 the main catheter 102 may include a plurality of segments 138 and (at least one) one or more pull wires 140 (for clarity, in FIGS. 19-21, the filter element 112 has been removed) in order to facilitate articulation and reconfiguration of the main catheter 102. More specifically, in the particular embodiment illustrated, the main catheter 102 includes at least one (e.g., a plurality of) inactive (passive) segments 138i and at least one (e.g., a plurality of) active (steerable, deflectable, articulable) segments 138a that are connected to a plurality of pull wires 140. The inactive segments 138i and the active segments 138a are arranged in a staggered pattern along a longitudinal axis X defined by the main catheter 102 such that the catheter 102 alternates between inactive segments 138i and active segments 138a. It is also envisioned that instead of one or more pull wires pulled proximally to bend/steer the active segment(s) or portion(s) of the catheter, one or more push wires can be provided along the catheter which can be pushed distally to bend/steer the active segment(s) or portion(s) of the catheter.

In the particular embodiment illustrated, each active segment 138a is connected to a corresponding (single) pull wire 140 that extends through (e.g., within) the outer wall 108 of the catheter 102 (e.g., such that the pull wires 140 are embedded within the outer wall 108), whereby the number of pull wires 140 corresponds to the number of active segments 138a. Upon the application of an axial (pulling) force to each of the pull wires 140, the corresponding active segment 138a is deflected (articulated) to thereby reconfigure (actively steer) the catheter 102 between a first (initial, normal) configuration (FIG. 19), in which the catheter 102 includes a (generally) linear configuration, and a second (subsequent, deflected) configuration (FIG. 21), in which the catheter 102 includes a non-linear configuration.

The use of a single pull wire 140 in connection with each active segment 138a reduces the requisite number of pull wires 140, thus reducing complexity in both construction and operation of the catheter 102. It is also envisioned that multiple, independently movable pull wires 140 may be included in other embodiments. In the particular embodiment illustrated, each pull wire 140 is received within a corresponding channel 142 (FIG. 20) that extends through the outer wall 108 of the catheter 102 in (generally) parallel relation to the longitudinal axis X.

To facilitate the application of axial force to the pull wires 140, in some embodiments, the catheter 102 may include (or may be connected to) a plurality of corresponding activating mechanisms 144 (e.g., such that the number of pull wires 140 corresponds to the number of activating mechanisms 144). In the particular embodiment illustrated, the catheter 102 includes a (first) activating mechanism 144i that is connected to the pull wire 140i and a (second) activating mechanism 144ii that is connected to the pull wire 140ii. The activating mechanisms 144 may include any structure or mechanism suitable for the intended purpose of applying the axial force to the pull wires 140 required to deflect the catheter 102 as necessary or desired, such as, for example, rotating wheels, pulley systems, or the like. In some embodiments, the active segments 138a, the pull wires 140, and the activating mechanisms 144 may be configured (and connected) such that each pull wire 140 may be individually acted upon to deflect (steer) the corresponding segment 138a in a single direction only. In other embodiments, pull wires 140 may be provided on various circumferential surfaces of the catheter 102 to facilitate steering in various directions.

In the particular embodiment illustrated, the catheter 102 includes a first inactive segment 138i1; a first active segment 138al that is located distally of the segment 138i1; a second inactive segment 13812 that is located distally of the segment 138al; and a second active segment 138a2 that is located distally of the segment 13812. Additionally, the catheter 102 includes respective first and second pull wires 140i, 140ii that are located within the channel 142 (FIG. 20). It is also envisioned, however, that the first and second pull wires 140i, 140ii may be located within separate channels 142 (e.g., such that the number of channels 142 corresponds to the number of pull wires 140).

The pull wires 140i, 140ii are connected to the segments 138a1, 138a2 at connection points 146i, 146ii (in addition to the activating mechanism 144i, 144ii), respectively, so as to facilitate reconfiguration of the catheter 102 between the first configuration (FIG. 19) and the second configuration (FIG. 21). More specifically, upon reconfiguration of the catheter 102, the active segments 138ai, 138aii define respective first and second bends 148i, 148ii (FIG. 21), which may be either substantially similar (e.g., identical) or dissimilar depending, for example, upon the particular configuration of the segments 138a1, 138a2, the materials of construction used in the catheter 102, the particular requirements of the catheter 102 dictated by the surgical procedure, etc. Although the bends 148i, 148ii are each illustrated as being (approximately) equal to 90 degrees in FIG. 21, depending upon the particular configuration of the segments 138a1, 138a2, the requirements of the surgical procedure, the particular anatomy of the patient, etc., it is envisioned that the bends 148i, 148ii may lie substantially within the range of approximately 0 degrees to approximately 270 degrees. For example, the segment 138al may be configured such that the bend 148i lies substantially within the range of approximately 0 degrees to approximately 180 degrees (e.g., approximately 90 degrees to approximately 180 degrees) and that the segment 138a2 may be configured such that the bend 148ii lies substantially within the range of approximately 0 degrees to approximately 270 degrees (e.g., approximately 90 degrees to approximately 270 degrees).

In the illustrated embodiment, the connection points 146i, 146ii are shown as being in (general) angular alignment (e.g., along a circumference of the catheter 102), which facilitates deflection of the segments 138a1, 138a2 in similar (e.g., identical) directions, as seen in FIG. 21. It is also envisioned, however, that the connection points 146i, 146ii may be angularly offset so as to facilitate deflection of the segments 138a1, 138a2 in dissimilar directions. For example, the connection points 146i, 146ii may be oriented in (generally) diametric opposition such that the bends 148i, 148ii respectively defined by the segments 138a1, 138a2 curve in (generally) opposite directions.

With reference now to FIG. 22, the main catheter 102 may include (at least one) one or more (second) pull wires 150 that are connected (secured, anchored) thereto, which may supplement or replace the pull wire(s) 140 (FIGS. 19-21) (for clarity, in FIG. 22, the filter element 112 has been removed). The pull wire(s) 150 facilitate the selective application of a torsional (twisting) force to the main catheter 102 and, thus, rotational deflection of the main catheter 102 along all or a portion of the length thereof to vary the angular position of the main catheter 102.

In contrast to the pull wires 140, the pull wire(s) 150 extend in non-parallel relation to the longitudinal axis X of the main catheter 102. In the particular embodiment illustrated, for example, the main catheter 102 includes a single pull wire 150 that is wound helically (spiraled) about the longitudinal axis X. It should be appreciated, however, that the number of pull wires 150 may be varied in alternate embodiments without departing from the present disclosure (e.g., it is envisioned that the main catheter 102 may include two pull wires 150, three pull wires 150, etc.). Such rotation, i.e., rotational deflection, via the torsional wire and steerability via other wires are disclosed in U.S. application Ser. No. 17/214,021 (U.S. Publication No. 2021/0259860) in conjunction with other systems, however, the features of the deflection are applicable to the catheters disclosed herein and the entire contents of the Ser. No. 17/214,021 (U.S. Publication No. 2021/0259860) application are incorporated herein by reference. Steerability features disclosed in PCT/US2022/051599 and PCT/US2021/023855 are also incorporated herein by reference in their entirety for use with the catheters disclosed herein.

It is envisioned that the pull wires 150 can fully or partially extend about the longitudinal axis X, i.e., extend 360 degrees, less than 360 degrees or greater than 360 degrees (more than one spiral).

In some embodiments, the pull wires 150 may include a (generally) linear configuration in a first portion of the main catheter 102 and a helical configuration in a second portion of the main catheter 102 (e.g., at the distal portion 106 of the main catheter 102) in order to effectuate rotational deflection of the second portion of the main catheter 102.

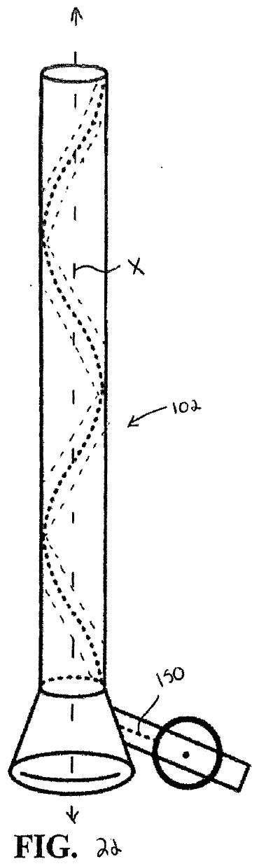

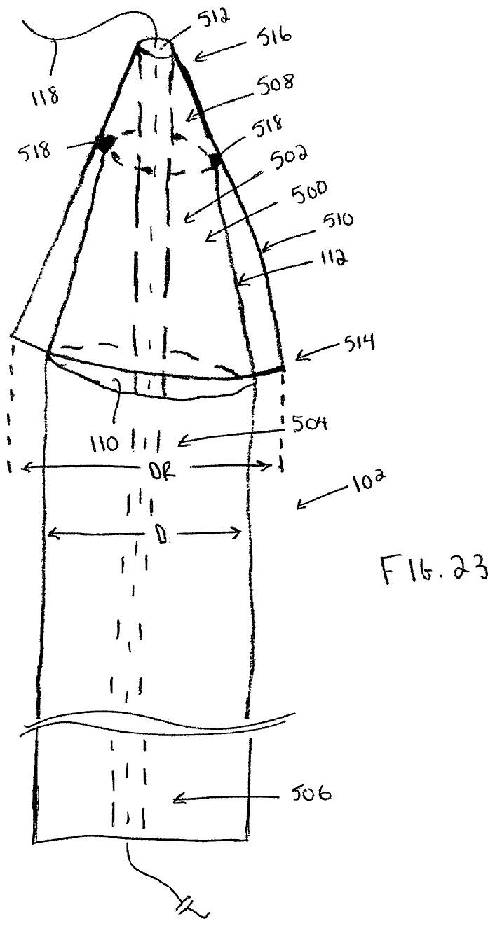

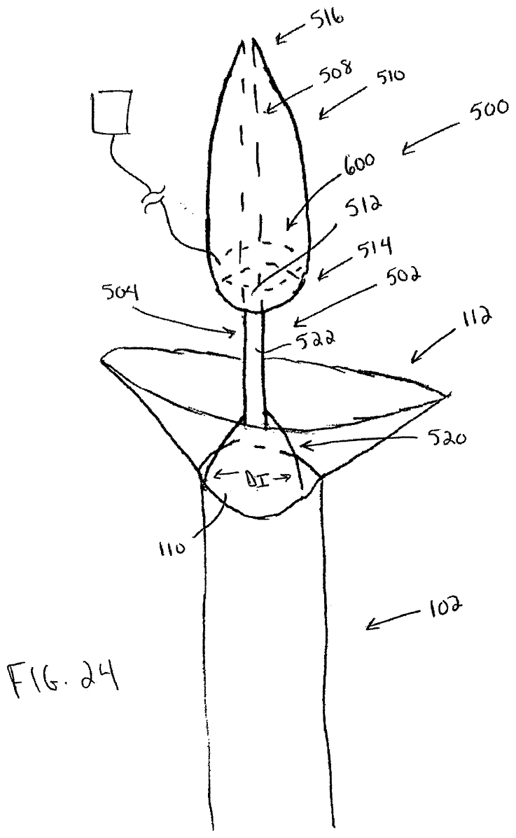

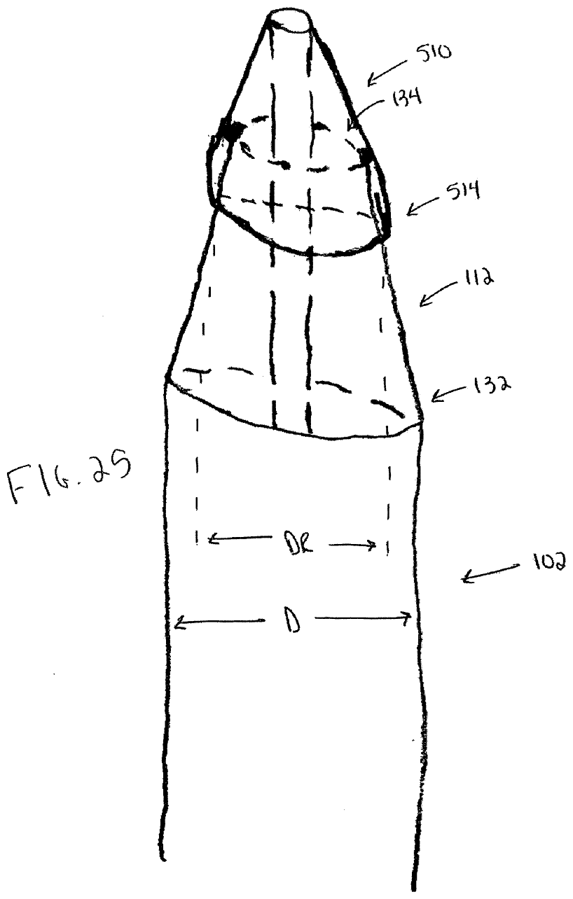

With reference now to FIGS. 23 and 24, in some embodiments, the endovascular system 10 may further include a (third) endovascular treatment (medical) device 500, which is configured as an inner (second) catheter (tubular member) 502. The inner catheter 502 may supplement or replace the outer sheath 300 (FIGS. 16, 17) and is configured to facilitate deployment of the filter element 112 and reconfiguration of the filter element 112 between the collapsed configuration and the expanded configuration (e.g., expansion and compression of the filter element 112), as described in further detail below.

The inner catheter 502 extends through the main catheter 102 (e.g., via the main lumen 110) and is (axially) movable in relation thereto between a retracted position (FIG. 23) and an advanced position (FIG. 24). The inner catheter 502 includes a body 504 having respective proximal and distal portions (ends) 506, 508 and a retainer 510 that is secured to the body 504. In certain embodiments, such as that which is illustrated, the body 504 of the inner catheter 502 defines a (central, working) lumen 512, which extends continuously between the respective proximal and distal portions 506, 508 thereof and is configured to receive the aforementioned guidewire 118 (FIG. 2) such that the inner catheter 502 (and the main catheter 102) are deliverable to the target treatment site through the blood vessel V (FIG. 1) over the guidewire 118.

The body 504 may include (at least one) one or more secondary or tertiary lumens.

Additionally, or alternatively, the body 504 may be configured to receive (at least one) one or more of the stylets 400 (FIG. 18) in order to further facilitate insertion of the inner catheter 502 into the blood vessel V.

Additionally, or alternatively, the body 504 may include (at least one) one or more steerable segments (zones) that are deflectable via (at least one) one or more pull wires and/or that the body 504 may be configured for rotational deflection to vary the angular position of the inner catheter 502, as discussed above in connection with the main catheter 102.

The retainer 510 includes a (free) proximal portion (end) 514 and a distal portion (end) 516 that is secured to the distal portion 508 of the body 504 such that the retainer 510 extends proximally therefrom. The retainer 510 includes a (generally) conical configuration and is configured to receive the filter element 112. More specifically, when the inner catheter 502 is in the retracted position (FIG. 23), the retainer 510 (e.g., the proximal portion 514 thereof) extends about the filter element 112 to thereby constrain the filter element 112 and preserve the collapsed configuration thereof (e.g., in order to facilitate delivery of the main catheter 102 and the filter element 112 to the target treatment site within the blood vessel V (FIG. 1)). When the inner catheter 502 is in the advanced position (FIG. 24), however, the retainer 510 is located distally beyond the filter element 112, which allows for (automatic) reconfiguration of the filter element 112 into the expanded configuration.

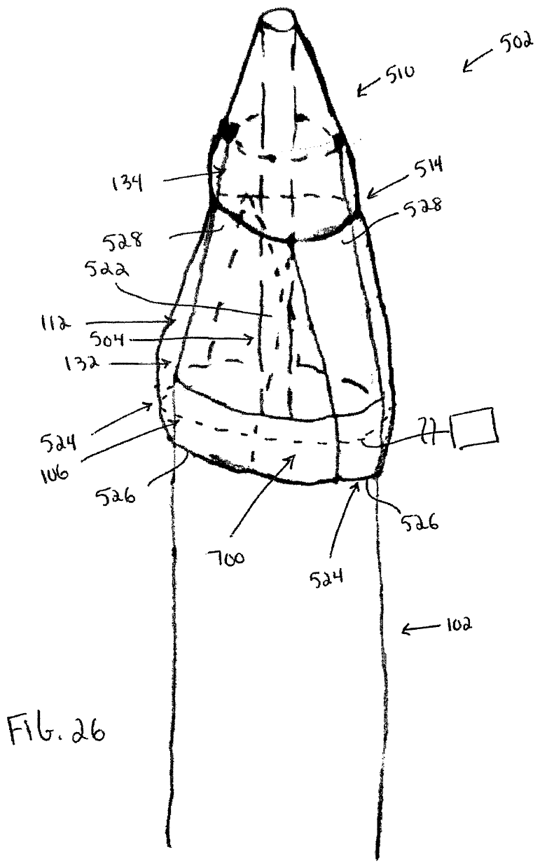

The retainer 510 may be configured to entirely conceal the filter element 112 in the retracted position, as seen in FIG. 23. Alternatively, however, the retainer 510 may be configured to partially conceal the filter element 112 in the retracted position (e.g., such that the proximal portion 514 of the retainer 510 is located axially between the respective proximal and distal portions 132, 134 of the filter element 112), as seen in FIG. 25.

Additionally, the retainer 510 may be configured such that the proximal portion 514 thereof defines an outer transverse cross-sectional dimension (e.g., a diameter) DR that exceeds the outer transverse cross-sectional dimension D of the main catheter 102. Embodiments are also envisioned, however, in which the retainer 510 may be configured such that outer transverse cross-sectional dimension DR may be less than the outer transverse cross-sectional dimension D, as seen in FIG. 25, as are embodiments in which the retainer 510 may be configured such that outer transverse cross-sectional dimension DR is (approximately) equal to the outer transverse cross-sectional dimension D.

In some embodiments, the retainer 510 may be connected to the main catheter 102 (e.g., the filter element 112) via (at least one) one or more frangible members 518, as seen in FIG. 23. The frangible member(s) 518 inhibit (if not entirely prevent) unintended relative movement between the inner catheter 502 and the main catheter 102 and, thus, unintended (e.g., premature) deployment (expansion) of the filter element 112. Upon the application sufficient (axial) force to the inner catheter 502 and/or the main catheter 102 (e.g., during movement of the inner catheter 502 from the retracted position (FIG. 23) into the advanced position (FIG. 24)), however, the frangible member(s) 518 are caused to rupture, which permits relative movement between the inner catheter 502 and the main catheter 102 and, thus, deployment (expansion) of the filter element 112. Embodiments in which the retainer 510 and the main catheter 102 (e.g., the filter element 112) are devoid of any (direct, fixed) connection therebetween are also envisioned herein, however, and would not be beyond the scope of the present disclosure.

While the frangible member(s) 518 are shown as being located axially between the respective proximal and distal portions 514, 516 of the retainer 510 in the illustrated embodiment, it should be appreciated that the frangible member(s) 518 may connect the retainer 510 to the main catheter 102 in any suitable location.

In some embodiments, the inner catheter 502 (e.g., the body 504) may include (at least one) one or more areas of increased thickness 520 (FIG. 24) such that the body 504 defines a transverse cross-sectional dimension (e.g., a diameter) D1 that is non-uniform between the respective proximal and distal portions 506, 508 thereof. The area(s) of increased thickness 520 increase the rigidity, strength, and/or the stability of the inner catheter 502 and, thus, support the application of (axial) force thereto during repositioning of the inner catheter 502 between the retracted position (FIG. 23) and the advanced position (FIG. 24). Embodiments in which the area(s) of increased thickness 520 may be omitted (e.g., embodiments in which the transverse cross-sectional dimension D1 may be (generally) uniform between the respective proximal and distal portions 506, 508 of the body 504) are also envisioned herein, however, and would not be beyond the scope of the present invention.

In some embodiments, the retainer 510 may be reconfigurable from an enlarged (first, non-constricted) configuration (FIG. 23) into a compressed (second, constricted) configuration (FIG. 24) during movement of the inner catheter 502 from the retracted position into the advanced position. More specifically, when the inner catheter 502 is in the retracted position, the retainer 510 is in the enlarged configuration such that the proximal portion 514 thereof and extends about the filter element 112 in order to maintain the filter element 112 in the collapsed configuration. When the inner catheter 502 is in the advanced position, however, the retainer 510 is in the compressed configuration and the proximal portion 514 thereof is spaced axially from the filter element 112.

In some embodiments, the retainer 510 may be biased towards the compressed configuration (e.g., radially inward towards the body 504 of the inner catheter 502) such that the retainer 510 is (automatically) reconfigured from the enlarged configuration (FIG. 23) into the compressed configuration (FIG. 24) upon movement of the inner catheter 502 from the retracted position into the advanced position in order to facilitate removal of the inner catheter 502 via proximal withdrawal through the main catheter 102. For example, it is envisioned that at least a portion of the retainer 510 (e.g., the proximal portion 514 thereof) may be resilient in construction. In such embodiments, upon movement of the inner catheter 502 into the advanced position, the proximal portion 514 of the retainer 510 may engage (contact, conform to) an outer surface 522 of the body 504 of the inner catheter 502, which further facilitates proximal withdrawal and removal of the inner catheter 502.

Additionally, or alternatively, the inner catheter 502 may include a cinching mechanism 600 (e.g., a lasso (snare) or the like) in order to facilitate reconfiguration of the retainer 510 from the enlarged configuration into the compressed configuration, as discussed above in connection with the filter element 112.

With reference now to FIG. 26, some embodiments, the inner catheter 502 may further include (at least one) one or more extensions 524. The extension(s) 524 include (free) proximal portion(s) (end(s)) 526 and distal portion(s) (end(s)) 528 that are secured to the proximal portion 514 of the retainer 510 (e.g., such that the distal portion(s) 528 of the extension(s) 524 are located axially between the respective proximal and distal portions 132, 134 of the filter element 112). The extension(s) 524 extend proximally from the retainer 510 so as to cover the filter element 112 and the distal portion 106 of the main catheter 102 when the inner catheter 502 is in the retracted position to further facilitate delivery of the main catheter 102 and the filter element 112 to the target treatment site within the blood vessel V (FIG. 1). When the inner catheter 502 is in the advanced position, however, the extension(s) 524 are spaced axially from the filter element 112 (e.g., such that the proximal portion(s) 526 of the extension(s) 524 are spaced distally from the distal portion 134 of the filter element 112).

In the illustrated embodiment, the inner catheter 502 includes a plurality of extensions 524 that are arranged in an overlapping, shingle-style configuration. Embodiments in which the inner catheter 502 may include a single extension 524 that extends circumferentially about the filter element 112 and the distal portion 134 of the main catheter 102 (e.g., in a continuous manner) are also envisioned herein, however, and would not be beyond the scope of the present invention.

In some embodiments, the extension(s) 524 may be connected to the main catheter 102 (e.g., the filter element 112) via (at least one) one or more of the aforementioned frangible members 518 (FIG. 23), which may replace or supplement those (releasably) connecting the retainer 510 to the filter element 112. In such embodiments, the frangible member(s) 518 may be positioned in any suitable location (e.g., such that the frangible member(s) 518 are located axially between the respective proximal and distal portions 526, 528 of the extension(s) 524). Embodiments in which the extension(s) 524 and the main catheter 102 (e.g., the filter element 112) may be devoid of any (direct, fixed) connection therebetween are also envisioned herein, however, and would not be beyond the scope of the present disclosure.

In some embodiments, the extension(s) 524 may be biased radially inward (e.g., towards the body 504 of the inner catheter 502) such that the extension(s) 524 are (automatically) deflected radially inward upon movement of the inner catheter 502 from the retracted position into the advanced position in order to facilitate removal of the inner catheter 502 via proximal withdrawal through the main catheter 102. For example, at least a portion of the extension(s) 524 (e.g., the proximal portion(s) 526 thereof) may be resilient in construction. In such embodiments, upon movement of the inner catheter 502 into the advanced position, it is envisioned that the proximal portion(s) 526 of the extension(s) 524 may engage (contact, conform to) the outer surface 522 of the body 504 of the inner catheter 502, which further facilitates proximal withdrawal and removal of the inner catheter 502.

Additionally, or alternatively, the extension(s) 524 may be deflected radially inward via the inclusion of a cinching mechanism 700 (e.g., a lasso (snare) or the like), as discussed above in connection with the filter element 112 and the retainer 510 (FIG. 24).

Additionally, or alternatively, in some embodiments, during proximal withdrawal of the inner catheter 502, it is envisioned that the extension(s) 524 may be inverted in order to facilitate removal of the inner catheter 502 from the main catheter 102. More specifically, in such embodiments, upon withdrawal of the inner catheter 502, the extension(s) 524 may be reconfigured such that the distal portion(s) 528 thereof are located distally beyond the distal portion 134 of the filter element 112.

In some embodiments, the catheter with the filter at the tip can be inserted into the ascending aorta via a transfemoral approach (or via other approaches) to deliver valves and other therapies with the filter providing protection from emboli. The catheter can optionally have an outer catheter that it is delivered through. When the filter is withdrawn into the outer catheter or the outer catheter is advanced over the filter, it collapses the filter. The outer catheter and/or the catheter with the filter at the tip can have at least one steerable zone controlled in the ways discussed above, e.g., controlled by at least one wire substantially in the wall or entirely in the wall of at least a portion of the respective catheter.

Persons skilled in the art will understand that the various embodiments of the invention described herein and shown in the accompanying figures constitute non-limiting examples, and that additional components and features may be added to any of the embodiments discussed herein above without departing from the scope of the present disclosure. Additionally, persons skilled in the art will understand that the elements and features shown or described in connection with one embodiment may be combined with those of another embodiment, including any of the embodiments described in U.S. patent application Ser. No. 17/210,778 (U.S. Publication No.: 2021/0236257) and 16/501,593 (U.S. Publication No.: 2020/0390015), without departing from the scope of the present invention and will appreciate further features and advantages of the presently disclosed subject matter based on the description provided. Variations, combinations, and/or modifications to any of the embodiments and/or features of the embodiments described herein that are within the abilities of a person having ordinary skill in the art are also within the scope of the invention, as are alternative embodiments that may result from combining, integrating, and/or omitting features from any of the disclosed embodiments.

In the preceding description, reference may be made to the spatial relationship between the various structures illustrated in the accompanying drawings, and to the spatial orientation of the structures. However, as will be recognized by those skilled in the art after a complete reading of this disclosure, the structures described herein may be positioned and oriented in any manner suitable for their intended purpose. Thus, the use of terms such as “above,” “below,” “upper,” “lower,” “inner,” “outer,” “left,” “right,” “upward,” “downward,” “inward,” “outward,” etc., should be understood to describe a relative relationship between the structures and/or a spatial orientation of the structures. Those skilled in the art will also recognize that the use of such terms may be provided in the context of the illustrations provided by the corresponding figure(s).

Additionally, terms such as “approximately,” “generally,” “substantially,” and the like should be understood to allow for variations in any numerical range or concept with which they are associated and encompass variations on the order of 25% (e.g., to allow for manufacturing tolerances and/or deviations in design). For example, the term “generally parallel” should be understood as referring to configurations in with the pertinent components are oriented so as to define an angle therebetween that is equal to 182°+25% (e.g., an angle that lies within the range of (approximately) 135° to (approximately)) 225° and the term “generally orthogonal” should be understood as referring to configurations in with the pertinent components are oriented so as to define an angle therebetween that is equal to 90°+25% (e.g., an angle that lies within the range of (approximately) 67.5° to (approximately)) 112.5°. The term “generally parallel” should thus be understood as referring to encompass configurations in which the pertinent components are arranged in parallel relation, and the term “generally orthogonal” should thus be understood as referring to encompass configurations in which the pertinent components are arranged in orthogonal relation.

Although terms such as “first,” “second,” “third,” etc., may be used herein to describe various operations, elements, components, regions, and/or sections, these operations, elements, 5 components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present invention. Also, the phrases “at least one of A, B, and C” and “A and/or B and/or C” should each be interpreted to include only A, only B, only C, or any combination of A, B, and C.

1. An endovascular system for insertion into a patient's blood vessel; the endovascular system comprising a first tubular member having a lumen and being configured for radial expansion to thereby enlarge the lumen upon insertion of a medical device and reconfigure the first tubular member from a first configuration, in which the main lumen defines a first dimension, into a second configuration, in which the lumen defines a second dimension larger than the first dimension.

2. The endovascular system of claim 1, wherein the first tubular member is substantially resilient in construction and includes at least one of rubber, latex or spandex.

3. The endovascular system of claim 1, wherein the first tubular member is configured to return to the first configuration following removal of the medical device.

4. The endovascular system of claim 1, wherein the first tubular member is reconfigured from the second configuration into a third configuration following removal of the medical device, the first tubular member defining a third dimension in the third configuration larger than the first dimension but smaller than the second dimension.

5. The endovascular system of claim 1, further comprising a filter element supported by the first tubular member and extending distally therefrom, the filter element including a semi-permeable membrane to allow blood cells and serum to pass therethrough while capturing emboli and being reconfigurable between a collapsed configuration for delivery and an expanded configuration within the patient's blood vessel, wherein at least a portion of the filter element includes a stretchable material to enable expansion of the filter element during passage of the medical device through the filter element.

6. (canceled)

7. The endovascular system of claim 5, further comprising a filter element supported by the first tubular member and extending distally therefrom, the filter element including a semi-permeable membrane to allow blood cells and serum to pass therethrough while capturing emboli and being reconfigurable between a collapsed configuration for delivery and an expanded configuration within the patient's blood vessel, wherein the filter element includes a proximal portion attached to the first tubular member and a distal portion, at least the proximal portion being expandable, wherein the proximal portion of the filter element includes a first material and the distal portion of the filter element includes a second material, wherein the second material is less resilient than the first material.

8-10. (canceled)

11. The endovascular system of claim 1, wherein the medical device includes a replacement valve.

12. The endovascular system of claim 1, wherein the first tubular member includes at least one steerable segment.

13. The endovascular system of claim 1, wherein the first tubular member is configured for rotational deflection to thereby vary an angular position of the first tubular member.

14. The endovascular system of claim 1, wherein the first tubular member recoils back to at least 50% of the first configuration after removal of the medical device from the lumen.

15. The endovascular system of claim 1, wherein the first tubular member recoils back to at least 90% 80% of the first configuration after removal of the medical device from the lumen.

16. The endovascular system of claim 1, wherein the first tubular member recoils back completely to the first configuration after removal of medical device from the lumen.

17-26. (canceled)

27. A catheter configured for use in treating vascular abnormalities, the catheter defining a lumen and including a filter element configured to capture emboli within a patient's blood vessel, the catheter being configured for radial expansion to thereby facilitate insertion of an endovascular treatment device into the lumen, whereby the catheter is reconfigured from a normal configuration, in which the lumen defines a first dimension, into an expanded configuration, in which the lumen defines a second dimension larger than the first dimension.

28. The catheter of claim 27, wherein the catheter includes at least one guide rail located within the lumen and configured for engagement with the endovascular treatment device to facilitate insertion of the endovascular treatment device into the catheter and advancement of the endovascular treatment device through the lumen.

29. The catheter of claim 27, wherein the catheter includes at least one strengthening rib configured to inhibit variation in an overall length of the catheter during radial expansion.

30. The catheter of claim 27, wherein the catheter includes a non-resilient construction such that the expanded configuration is generally maintained following removal of the endovascular treatment device.

31. The catheter of claim 1, wherein the first tubular member includes a resilient construction such that the first tubular member is biased towards the normal configuration, wherein the first tubular member includes at least one stretchable material, the stretchable material being a stretchy resin comprising a braid of wires, non-metal fibers or a combination of wires and non-metal fibers.

32. (canceled)

33. The catheter of claim 27, wherein the catheter is configured to return to the normal configuration following removal of the endovascular treatment device.

34. The catheter of claim 27, wherein the catheter is configured such that, following removal of the endovascular treatment device, the catheter is reconfigured into a partially-expanded configuration in which the lumen defines a third dimension larger than the first dimension and smaller than the second dimension.

35-54. (canceled)

55. The endovascular system of claim 27, wherein the catheter recoils back at least 80% of its initial configuration after an endovascular treatment device inserted to expand the lumen is removed.

56-62. (canceled)

63. The endovascular system of claim 31, wherein the braid protrudes along an inner diameter of the first tubular member to act as rails for the endovascular treatment device to slide on to minimize friction along the surface of the first tubular member.

64. The endovascular system of claim 31, wherein the braid changes configuration without the fibers elongating.

65. The endovascular system of claim 1, further comprising an inner hydrophilic coating.
